(19) United States
(12) Patent Application Publication
(10) Pub. No.: US 2010/0036394 A1
(43) Pub. Date: Feb. 11, 2010

(54) Magnetic Levitation Based Devices, Systems and Techniques for Probing and Operating in Confined Space, Including Performing Medical Diagnosis and Surgical Procedures

(76) Inventors: Yoav Mintz, Jerusalem (IL); Martin Simon, Los Angeles, CA (US)

Correspondence Address: FISH & RICHARDSON, PC, P.O. BOX 1022, MINNEAPOLIS, MN, 55440-1022, US

(21) Appl. No.: 12/525,498

(22) PCT Filed: Jan. 30, 2008
(86) PCT No.: PCT/US08/52466

§ 371 Date: Aug. 17, 2009

Related U.S. Application Data
(60) Provisional application No. 60/887,548, filed on Jan. 31, 2007.

(51) Int. Cl. A61B 19/00

(52) U.S. Cl. 606/130

(57) ABSTRACT

Apparatus, systems and techniques for implementing a servo controlled magnetic levitation system that magnetically levitates and controls a magnetic platform to navigate in a confined space to obtain capture images of or other information of the confined space. Medical surgical or diagnostic systems and various detection systems may be constructed based on the described systems.

This patent application claims the benefit of U.S. Provisional Application No. 60/887,548 entitled “MAGNETIC LEVITATION BASED DEVICES, SYSTEMS AND TECHNIQUES FOR PROBING AND OPERATING IN CONFINED SPACE, INCLUDING PERFORMING MEDICAL DIAGNOSIS AND SURGICAL PROCEDURES” and filed on Jan. 31, 2007, the disclosure of which is incorporated by reference as part of the specification of this application.

### BACKGROUND

This application relates to magnetic levitation based devices, systems and techniques for various applications including medical diagnosis and surgery applications.

Magnetic levitation techniques apply a magnetic field to levitate or suspend a magnetic object based on the interaction between the magnetic object and the applied magnetic field. The magnetic field is designed in a way so that the interaction counteracts other forces exerted on the object such as the gravitational force. When the magnetic object is a paramagnetic or ferromagnetic, stable magnetic levitation requires the levitated magnetic object to be placed at a location where the levitating magnetic field has a maximum. Because a magnetic field in free space cannot have a maximum, stable magnetic levitation is impossible for a paramagnetic or ferromagnetic object. This is known as the Earnshaw's theorem.

Stable magnetic levitation can be achieved, however, when a levitation system is designed to violate the conditions for the Earnshaw's theorem. For example, a diamagnetic material can be levitated and stabilized. See, “Magnet levitation at your fingertips” by A. K. Geim, M. D. Simon, M. I. Boamfa, and L. O. Heflinger in Nature, vol. 400, p. 323-324 (1999); “Diamagnetically stabilized magnet levitation” by M. D. Simon, A. K. Geim and L. O. Heflinger in America Journal of Physics, Vol. 69(6), p. 702-713 (2001). A magnetic object can also be levitated and stabilized in a magnetic system with an electronic feedback control to dynamically adjust one or more electromagnets in the system to stabilize the magnetically levitated object at a desired location. One example of a dynamically controlled magnetically levitated system is the electromagnetic suspension (EMS) magnetic levitation train where a servo control system adjusts a magnetic force at a constant distance from the track.

### SUMMARY

The specification of this application describes, among others, embodiments and implementations of techniques, apparatus and systems for implementing a servo controlled magnetic levitation system that magnetically levitates and controls a magnetic platform to navigate in a confined space to obtain capture images of or other information of the confined space. A servo control is provided to control the magnetic field that levitates the magnetic platform to stabilize the levitated magnetic platform. The magnetic platform can be equipped with instrumentation to perform other operations in the confined space. The confined space may be a location with a chemical or biological hazard substance so the magnetic platform can be used to detect such substance. In medical applications, the magnetic platform can be inserted into a patient's body to perform medical diagnosis or surgery with a significantly reduced level of incisions.

In one embodiment, for example, a magnetic levitation system can be implemented to include a frame that includes magnets to produce a variable magnetic field, magnetic field sensors mounted to the frame to measure the magnetic field, a magnetic platform formed of a magnetic material and configured to be magnetically levitated by the magnetic field, and a feedback control module to receive sensor signals from the magnetic field sensors and, in response to the sensor signals, to control the magnets to adjust the magnetic field to levitate and to stabilize the magnetic platform. The magnetic platform includes a video camera to capture video images and a wireless communication unit to wirelessly transmit the video images outside the magnetic platform.

In another embodiment, for example, a magnetic levitation medical system includes a frame structured to define a surgical space that accommodates a surgical table for holding a patient, the magnetic frame comprising a top frame part above the surgical space. This system includes a magnet system which includes (1) a plurality of lifting magnets mounted to the top frame part to produce a static magnetic field within the surgical space below the top frame part to exert a magnetic lifting force on a magnetic material against the gravity, and (2) at least one electromagnet mounted to the top frame part to produce an adjustable magnetic field in the surgical space. Magnetic field sensors are mounted to the frame to measure the magnetic field in the surgical space. This system also includes a magnetic platform formed of a magnetic material and configured to be magnetically levitated by the magnet system to levitate in the surgical space without a mechanical attachment and without a communication cable. The magnetic platform includes at least one of a diagnostic probe that performs a measurement and a surgical tool that performs a surgical operation. A feedback control module is provided to receive sensor signals from the magnetic field sensors and, in response to the sensor signals, to control the magnet system to adjust the magnetic field in the surgical space to stabilize and to control a position and motion of the levitated magnetic platform.

In yet another embodiment, a method is described for operating a magnetically levitated platform to conduct a surgical operation within an abdominal cavity of a patient. This method includes providing a magnet system to produce a variable magnetic field that defines a magnetic levitation region above a surgical table to levitate a magnetic platform and to control a position and motion of the magnetic platform; placing the patient on the surgical table to position the abdominal cavity in the magnetic levitation region; inflating the abdominal cavity with a gas; and inserting the magnetic platform inside the inflated abdominal cavity to levitate the magnetic platform. Next, this method provides controlling the variable magnetic field in the magnetic levitation region to levitate the magnetic platform in the inflated abdominal cavity and to stabilize the magnetic platform; using a camera on the magnetic platform to capture one or more images of inside the inflated abdominal cavity including the target; and wirelessly transmitting the one or more captured images from the camera outside the patient's body to display on a display screen. This method further provides moving the relative position or orientation of the magnet system with respect to the surgical table to move the levitated magnetic platform near a target area within the inflated abdominal cavity; and wirelessly controlling a surgical instrument mounted on the levitated magnetic platform to perform a surgical operation on the target area within the inflated abdominal cavity.

These and other examples and implementations are described in detail in the drawings, the detailed description, and the claims.

### BRIEF DESCRIPTION OF THE DRAWINGS

FIG. 1 shows one example of a magnetic levitation system for medical applications.

FIG. 2 illustrates a levitated magnetic platform inside an inflated cavity within a patient.

FIG. 3 shows an example of a control console for the system in FIG. 1.

FIGS. 4 and 5 illustrate examples of control of the magnetic levitation in the system in FIG. 1.

FIG. 6 shows an example of a magnetic levitation system having an adjustable operation table.

### DETAILED DESCRIPTION

The magnetic levitation apparatus, systems and techniques described in this application may be used in medical surgical and diagnostic applications, detection of a hazardous condition, chemical or biological substances and other conditions in confined space, and other applications. One example of the present magnetic levitation systems can include a frame comprising magnets to produce a variable magnetic field, magnetic field sensors mounted to the frame to measure the magnetic field, a magnetic platform formed of a magnetic material and configured to be magnetically levitated by the magnetic field, and a feedback control module to receive sensor signals from the magnetic field sensors and, in response to the sensor signals, to control the magnets to adjust the magnetic field to levitate and to stabilize the magnetic platform. The magnetic platform includes a video camera to capture video images and a wireless communication unit to wirelessly transmit the video images outside the magnetic platform.

Implementations of this and other magnetic levitation systems described in this application may be configured for medical and surgical uses. The following examples provide some details for specific medial surgical and diagnostic uses in which the magnetic platform is levitated and is remotely controlled and inserted into hard to reach cavities and spaces. While in these spaces certain functions could be performed according to the tasks needed. The basic function is transferring images such as live streaming video images of that space and further functions can include handling its surrounding i.e. moving parts, repairing, achieving biopsies or samples or performing surgery. For medicine use, this platform can be inserted to a cavity of a subject, e.g., the peritoneal cavity, the gastrointestinal tract, the nasopharyngeal space, and the oral cavity.

Such a magnetic levitation system can be used to provide Minimal Invasive Surgery (MIS) in areas such as the abdominal and chest regions. MIS has gained a major role in the various surgical procedures during the last decade. Laparoscopic cholecystectomy is possibly the most common laparoscopic operation and on 2003 between 500,000 to 600,000 laparoscopic cholecystectomies were performed in the US, which comprised 25% of the operations in general surgery that year. Since then laparoscopic surgery has become even more common (on 2003 the CDC estimated that 750,000 operations were performed in the US) and today minimal invasive surgery is the preferred type of surgery in many types of operations.

MIS operations can be performed in various ways. For example, small incisions in the abdominal wall can be made to perform MIS operations. As a specific example, the first step of the MIS operation can be to inflate the abdominal cavity with a gas (e.g., the CO2 gas) in order to create a working space. Then, several 5-12 mm skin incisions are performed and trocars (plastic or metallic sheaths which serve as ports of entry) are inserted into the abdominal cavity through these incisions. The laparoscope (camera attached to a shaft with lenses and fiber optic cables) is inserted through one of the trocars and enables the vision of the abdominal organs on a TV monitor placed beside the patient. Insertion of long instruments through the other trocars follow, which enable performing the surgery. Among the major advantages of MIS are the small incisions that cause less trauma to the abdominal wall and hence a lesser degree metabolic response to trauma. Also the small incisions cause less post operative pain and therefore less respiratory complications. The operation itself is performed through a magnified image with enhanced vision and lighting which enables operating in deep abdominal space without difficulty. Due to the minimal abdominal trauma and reduction of post operative pain the length of hospital stay decreased significantly. The cosmetic results can be better than some other surgery procedures that have been used by surgeons.

In some implementations, the above described MIS operations can have some disadvantages. For example, the operation is performed using two dimensional vision on the TV monitor and such vision can limit the precision of the operation. Such operation also lacks the tactile sensation and thus is limited in precision and other aspects. The operating field can be restricted to the camera field of view. Such operations tend to involve certain counter intuitive movements and thus may require special expertise. As yet another example, in such operations, the field of the operation is usually restricted by the choice of the primary incisions.

In an operation by the MIS methods, inserting instruments through the abdominal wall may create a hinge for the movements of these instruments. The hinge is located where the incision is made and hence the instruments may experience restricted movement which is determined by the choice of the incision location. The approach to an organ in the abdomen is determined by the point of entry on the abdominal wall and the point of contact with that organ. Because these two points define one straight line, these two points dictate the direction along which this organ can be approached. If the surgeon wants to approach the organ from a different angle he needs to make another incision on the abdominal wall and insert the instrument from there. Currently the camera used for MIS is attached to a shaft comprised of lenses which convey the image from within the abdomen. The same restriction applies to the camera. The surgeon may not always be able to view the organs from a desired angle and this condition can compromise the vision capabilities in making further incisions.

A magnetic levitation system described in this application can be applied to MIS operations and to address one or more issues discussed above. A magnetic levitation medical system include, for example, a frame structured to define a surgical space that accommodates a surgical table for holding a patient, the frame comprising a top frame part above the surgical space; a magnet system which includes (1) lifting magnets mounted to the top frame part to produce a static magnetic field within the surgical space below the top frame part to exert a magnetic lifting force on a magnetic material against the gravity, and (2) at least one electromagnet mounted to the top frame part to produce an adjustable magnetic field in the surgical space; and magnetic field sensors mounted to the frame to measure the magnetic field in the surgical space. In some implementations, at least one of the magnetic field sensors may be mounted to the top frame part. This system also includes a magnetic platform formed of a magnetic material and configured to be magnetically levitated by the magnetic system to levitate in the surgical space without a mechanical attachment and without a communication cable. This magnetic platform includes at least one of a diagnostic probe that performs a measurement and a surgical tool that performs a surgical operation. The sensor signals generated from the magnetic field sensors can be used to extract position information of the magnetic platform. Other position sensors different from the magnetic field sensors may also be implemented. This system further includes a feedback control module to receive sensor signals from the magnetic field sensors or other position sensors and, in response to the sensor signals, to control the magnet system to adjust the magnetic field in the surgical space to stabilize and to control a position and motion of the levitated magnetic platform.

Having the magnetic platform that levitates in the abdominal cavity provides a number of advantages. For example, this platform can change its location within the cavity by remote control and hence achieving the desired angle of approach to the organs without adding further incisions on the abdominal wall. The levitating platform can be equipped to perform different tasks. For example the basic device would be a miniature wireless camera. This camera may include two imaging sensors such as two CMOS sensors as to achieve a 3D vision and will transmit live video images to the outer portion of the cavity. A computer processor can be used to process the captured images to enhance the images and allow the 3D vision for the operator. In order to visualize inside a dark cavity as the abdomen a LED light source and a DC power source may be harbored on the platform as well.

In some implementations, the magnetic platform can incorporate selected Robotic surgery (MIS using robotic assistance) mechanisms. A laparoscope used in robotic surgery, for example, can be implemented on the magnetic platform to enable 3 dimensional vision; in another example, computer software can be used to enable tremor elimination and fine tuning of the surgeons movements and the counter-intuitive movements are translated automatically by the robot to the more friendly intuitive movements.

In other MIS surgical operations, it can be difficult to overcome the restriction of the field of vision by the placements of the trocars, and the restriction of the direction of the working instruments. The lack of possibility to visualize the side part of the organ operated upon and the need for a “blind” dissection of tissues due to that fact poses a major disadvantage to MIS and robotic surgery. As long as the camera and the working instruments are inserted through trocars, the hinges created at the abdominal wall can restrict their movement and direction of manipulation inside the abdominal cavity.

The present magnetic levitation technology can be configured in ways that mitigate these and other issues using the levitated magnetic platform which is remotely controlled and acts as a medical diagnosis and surgical device vehicle. The platform can harbor a video camera which transmits live streaming video images via wireless connection from within the cavity to a receiver placed outside of the cavity. These images are viewed on a monitor or a 3D visor when a 3D camera is used. According to the images acquired, the movement of the platform is controlled, in real time, by the operator according to his commands, on matching “joysticks” in the console. The platform can be moved in 3 axis directions in relation to the cavity by moving the lifter magnet complex or moving the cavity itself. The platform can be used as the only tool for the procedure when harbored with a camera, light source and surgical instruments or as an adjunct when harbored with only a camera to aid in the visualization or only instrumentation to perform the procedure when achieving video images from another source. Among various advantages and benefits, such a magnetic levitation system can be implemented in a way to perform the procedure having a better vision and better access to the organs with fewer incisions on the abdominal wall.

The instruments harbored on the platform could be moved as one part of the platform or independently relatively to the platform. In one example, the operating instruments can be specially constructed using MEMS technology which enables the instruments to be small, receive wireless signals interpret them and translate them into the ordered movements.

Various magnetic levitation systems and techniques are known and have been applied in various areas of industry and in the toys industry. In some systems, the levitation gap used is of a few millimeters to a few centimeters mostly in order to accelerate speed of rotating/moving parts and diminishing the friction. The systems described in this application can be implemented by using dynamically controlled feedback to control the magnetic fields and to stabilize the levitated platform with a gap of tens of centimeters, e.g., 10-20 cm, to provide sufficient space for medical surgical and diagnostic operations through instrumentation on the levitated platform. Magnetic position sensors and feedback control of the magnets provide remote control of the levitated platform.

The present magnetic levitation systems and techniques can be applied to other applications, including but not limited to, handling of hazardous materials (radioactive substances, dangerous gaseous materials, highly infectious organisms), and gentle mechanical work in confined areas or outer space. Such applications can allow for minimal human contact and provide maximal precision. These remotely controlled systems may be operated from a distance further than a few meters, by internet connections. This way expertise of certain procedures could be shared worldwide with out the necessity to transfer patients, physicians or other expert personnel across countries.

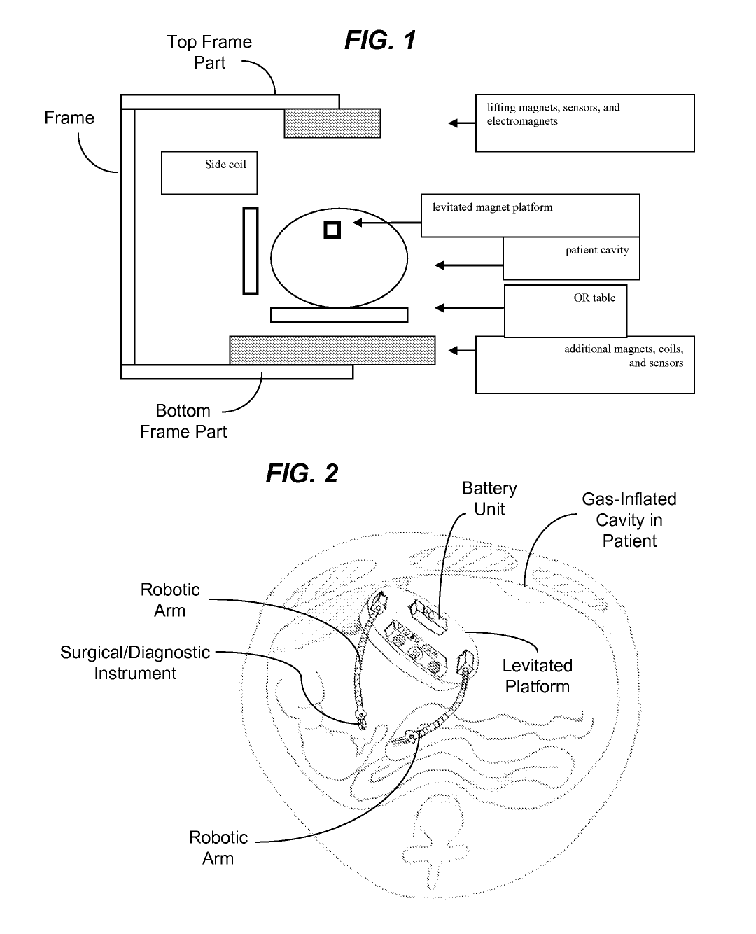

FIG. 1 illustrates one example of a magnetic levitation system for medical surgical and diagnostic operations. In this example, a frame for supporting various components includes a top frame part that is structured to hold lifting permanent magnets, magnetic sensors and electromagnets. The magnets are positioned and controlled to produce a variable magnetic field in a surgical space. The frame may include a bottom frame part which holds additional magnets, electromagnets and magnetic sensors. The frame may further include at least one side frame part that is positioned vertically below the top frame part and at one side of the surgical space, and at least one electromagnet mounted to the side frame part to exert a side adjustable magnetic field in the surgical space to provide an additional control over the levitation of the magnetic platform.

The total magnetic field produced at the surgical space can magnetically levitate the magnetic platform with a large gap with its surroundings so that it can be moved inside a cavity of a patient placed on a surgical table. One or more electromagnets can be used to create the appropriate time changing magnetic field and the magnetic field sensors are used to measure the position and motion of the platform. A control circuit can be used to keep the platform stably levitating by controlling the current to one or more electromagnets or the field shaping magnets. Motion control for the subject cavity or for the field shaping magnets can also be provided as part of the system control to move the levitated platform. The levitated platform can be controlled and positioned in a gas-filled or vacuum cavity. The large gap, that is, the large clearance volume above, below, and to the sides of the levitating platform is meant to accommodate the human body undergoing a laparoscopy procedure, heart/lung or other surgery. For example, the levitated platform can be controlled at the center of a one cubic foot volume with no part of the levitation apparatus closer than 6 inches in any direction. The magnet platform can be configured to fit into the cavity opening and structured to support instrumentation for medical surgery or diagnostic measurements.

The field shaping magnets are arranged so that the magnetic field has the proper direction, gradient and curvature at the desired levitation point where the platform levitates. The field direction keeps the platform from overturning and the gradient is set at an appropriate value and along an appropriate direction to balance out the gravity at the levitation point. The magnetic field curvature determines which directions are stable for the platform and which directions are unstable. The feedback control is used to control magnetic field that levitates the platform to stabilize the platform. Various configurations for the field shaping coils are possible. As an example, the system in FIG. 1 can be configured to have the instability and feedback control restricted to the vertical direction. The magnetic sensors can be used as position sensors to measure the position of the platform in the levitating magnetic field relative to a stable levitation position in the levitating magnetic field. The velocity of the platform can also be derived from the sensor signals of the same magnetic sensors because the velocity is the rate of change in position or differential of the platform position. When the platform moves from the desired position the magnetic position sensor and circuit generates an error signal proportional to how far it has moved. The differential signal is processed in an analog or digital way to derive the velocity which generates the differential error signal which is amplified and added to the position error. The PID (proportional, integral, and differential) controller uses the combined error signal to control the electromagnet current to reduce the error and keep the platform at the desired position.

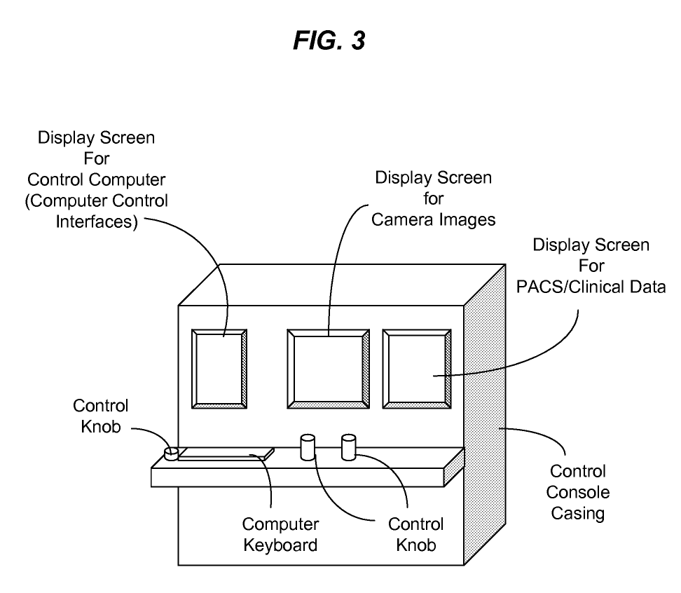

FIG. 2 illustrates a levitated magnetic platform in a gas-inflated cavity of a patient. The magnetic platform in this example is shown to include a portable power unit such as a battery unit to supply the power, robotic arms equipped with instrumentation for medical surgery or diagnostics and a video camera for capturing images inside the cavity. The robotic arms and surgical and diagnostic tools on the platform can be made from non-magnetic materials so that their operations are not affected by the presence of the levitation magnetic field.

FIG. 3 shows an example of a control console that includes a computer display for showing computer-user interface, an image display for showing images (e.g., 3D images) from the camera on the platform, and a third display for showing various medical data and charts such as picture achieving and communication systems (“PACS”) for medical applications and clinical data including data for the patient. The control console can include one or more user interface input and output devices such as a keyboard, a computer pointing device (e.g., a mouse) and control knobs such as joysticks for navigating the position and motion of the platform, controlling the robotic arms on the platform, and other operations. The console is used to control and monitor the operations of the magnetic levitation system.

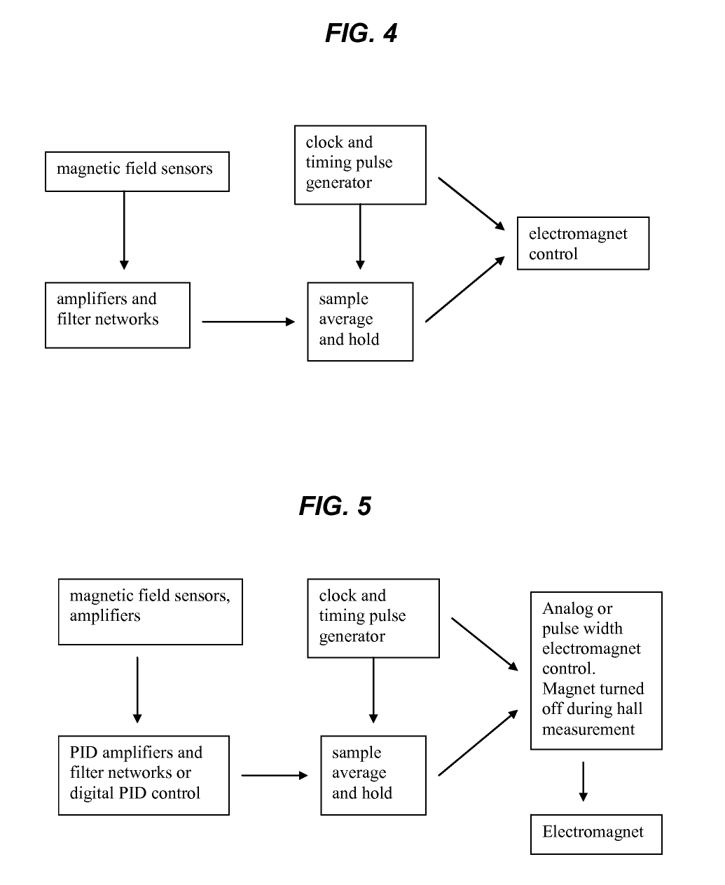

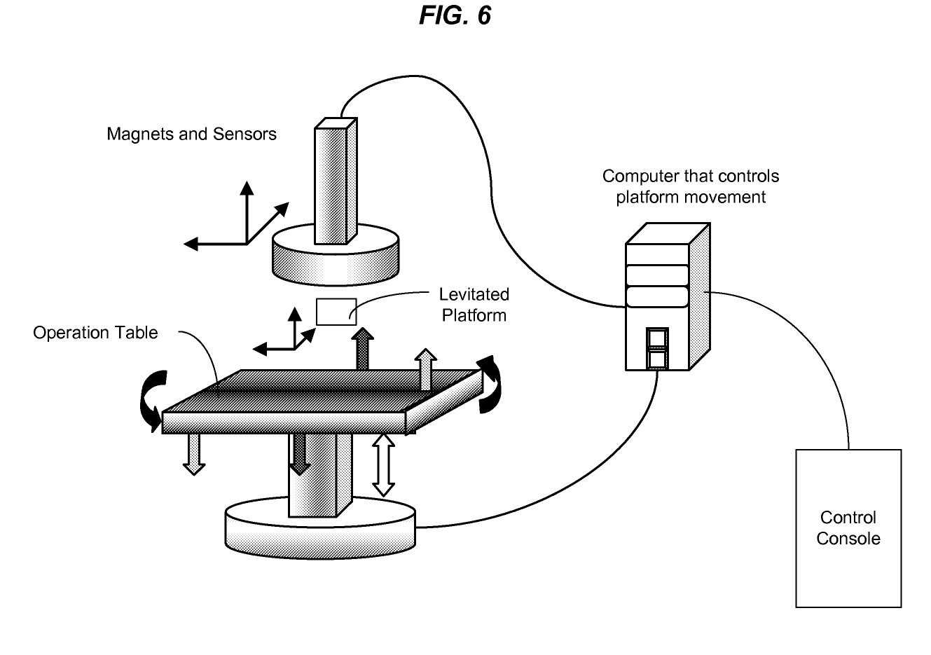

FIG. 4 illustrates one example of a control circuitry for controlling the magnets to levitate and control the magnetic platform. A magnetic field sensor can be located at a remote position from the payload platform and can be configured to operate without requiring a line of sight communication (e.g., an optical path) so that the payload can operate in an enclosed cavity. The signal is processed through a series of amplifiers and filters and then brought to a sample and hold amplifier. The sample and hold module can repeat the operation over 1000 times a second under the control of timing pulses from the timing pulse generator circuit. The timing circuit can shut down the electromagnet before the sample and hold starts averaging, and turn the electromagnet back on after the hold value is latched. This circuit design can increase the signal to noise ratio of the remote magnet platform position sensor with a large clear volume gap. Subtracting sensors and current sensors can also be used to reduce noise.

The magnets in FIG. 1 and the control mechanism can be designed to provide a magnetic field geometry that moves the levitation point from less than, e.g., one inch away from a coil to more than 5 inches away from the coil or any part of the structure. The field is designed to levitate the magnet platform and a payload at a desired position. The magnetic sensor can also be designed to be away from the levitated platform at a large distance (e.g., at least 5 inches). Since magnetic fields fall off by approximately the cube of the distance, the signal to noise ratio can be more than 100 times smaller than other maglev systems where the levitated object is much closer to the magnets and sensors. The system can be designed and controlled to move the platform in three dimensions with respect to the cavity.

FIG. 5 shows an example implementation of the control in FIG. 4. The control circuit can be a PID controller, which stands for proportional (P), integral (I), and derivative (D) controller. The signal from a Hall effect magnetic sensor represents the position and orientation of the levitated magnet platform, after the fields from the permanent magnet array on the head are subtracted out. The pulsing of the electromagnet is also seen by the Hall probe and this effect can be calibrated in processing the sensor signals. By various means, the signal from the Hall probe is taken directly, (the proportional part of the PID), differentiated and amplified (the D part), and integrated and amplified (the I part). The proportional signal involves the distance the platform has moved away from the desired position and is the error signal that indicates the deviation of the platform from a position where the levitation is stabilized. The differential signal represents the velocity of the platform. These signals are used to control the electromagnet in such a way as to reduce the position error. The PID controller can be implemented by analog electronics as described above, or in a more compact way by a phase lead network. It can also be implemented in digital form, where the signal from the hall sensor is digitized and then processed numerically by a computer. The computer uses a transfer function to modify the signal in the same way the filters and amplifiers of the analog circuit do. The computer could be a desktop or laptop computer with an input and output device or it could be a programmable circuit (e.g., a 20 pin chip) that may be mounted on a circuit board with the rest of the electronics. A 20-pin 8-bit microprocessor programmed in assembly language is an example for the digital processor for the above control.

Two electromagnet controls may be applied in implementations and other methods are also possible. The first is analog control, where the amplitude of the electromagnet current is varied. The second is pulse width modulation where the current is driven on full each time but the width of the pulse is varied. One design uses a bipolar system where the coil can be driven with positive or negative current but unipolar designs are also possible. Both amplitude and pulse width modulations can be used.

Some technical challenges in implementations include: large separation between the magnet platform and the hall sensor may cause the error signal to be smaller than the noise in the hall sensor; pulsing of the control coil may rail the Hall signal from each magnetic sensor. To overcome these problems, a sample, average, and hold scheme can be implemented in analog circuitry, in digital processing (e.g., via processing software) or a combination of analogy circuitry and digital processing. The electromagnet is turned off for a brief period of time. During this time the Hall sensor signal is put through a set of filters (analog or digital) and amplifiers, and the output is averaged and then the average value is latched. The generated error value is used to set the amplitude of the electromagnet current for a set period of time. This process is repeated (e.g., over 1000 times a second). The signal noise is reduced as the averaging time increases. The averaging time is limited below a certain value to keep the response of the control sufficiently fast. The control can be implemented as a phase lead network or analog or digital PID controller.

There are several options of controlling the platform movement depending on configurations of the lifter magnets and magnetic sensors. The following are some examples.

In a configuration that the lifter magnets are above the OR table and the sensor is at the same place, the platform movement can be controlled by moving the lifter magnet complex. The operator can use a control device such as a joystick on the control console in FIG. 3 to move the platform. Computer software in the control console can be used to translate the joystick movements performed by the operator to the lifter magnet complex movement. Any movement of the joystick causes in fact movement of the lifter magnet complex (not necessarily 1:1). The levitated platform is locked in the magnetic field created by the lifter magnets and hence it follows the movement of the lifter magnet complex. This movement can be achieved in the 3 dimensions by elevation of the complex and forward and sideways movements in the horizontal plane. Rotation of the platform can be achieved as well due to the asymmetric design of the platform. The lifter magnet complex could be ceiling mounted on a moving platform or on a mobile cart positioned above the OR table.

In a configuration that the sensors are not positioned in the lifter magnet complex the sensor is moving synchronously with the lifter magnets using the same computer software for both complexes (lifter magnets and sensors).

A different way to achieve the relative movement of the platform to the patient is by moving the patient while the platform is stationary. The patient moves due to the joystick movements that control the movement of the OR table via computer control software in the system. The lifter magnet complex stays stationary and therefore the levitated platform as well. In this way the movement of the platform is relative to the patient and achieves the same result. This is a lesser preferred way due to the necessary movement of the anesthesia equipment. The organs might move as well due to the changes of gravity forces while tilting the table in the horizontal plane.

A third way of controlling the platform movement is a combination of the lifter magnet complex movement and the OR table movement. In this configuration the default is moving the lifter magnet complex. If necessary the computer software can be used to add the additional distance desired by moving the OR table.

An example flow of operations for operating a patient using the magnetic levitation system is now described. First, the patient is positioned on the OR table and anesthesia, prep and drape are performed. The surgery begins with a small incision (e.g., a 2.5 cm cut) on the abdominal wall at a suitable location which can be selected based on the patient's specific conditions and requirements such as previous scars, previous surgery and cosmetic results. The designated laparoscopic instrument is then inserted through the trocar and hold the platform with the lap instrument. The platform is then inserted into the abdominal cavity through the incision. Next, the fascial incision is sutured tight around the trocar and subsequently the peritoneal cavity is filled with a gas (CO2) to inflate, thus forming the working space as in laparoscopic surgery.

At this time, the magnets of the system are controlled to levitate the platform inside the cavity and to stabilize and lock the platform in the magnetic field. The medical surgery or diagnostic procedure is then performed using the levitated platform. Following the termination of the procedure, the levitated platform is moved to the incision site. Insert the lap instrument and hold the platform. Next, the levitation magnetic field is shot down to remove the platform through the incision. Finally, the surgeon sutures the incision to complete the procedure.

Hence, in one implementation, a method for operating a magnetically levitated platform to conduct a surgical operation within an abdominal cavity of a patient can include: providing a magnet system to produce a variable magnetic field that defines a magnetic levitation region above a surgical table to levitate a magnetic platform and to control a position and motion of the magnetic platform; placing the patient on the surgical table to position the abdominal cavity in the magnetic levitation region; inflating the abdominal cavity with a gas; and inserting the magnetic platform inside the inflated abdominal cavity to levitate the magnetic platform. Next, this method provides controlling the variable magnetic field in the magnetic levitation region to levitate the magnetic platform in the inflated abdominal cavity and to stabilize the magnetic platform; using a camera on the magnetic platform to capture one or more images of inside the inflated abdominal cavity including the target; and wirelessly transmitting the one or more captured images from the camera outside the patient's body to display on a display screen. This method further provides moving the relative position or orientation of the magnet system with respect to the surgical table to move the levitated magnetic platform near a target area within the inflated abdominal cavity; and wirelessly controlling a surgical instrument mounted on the levitated magnetic platform to perform a surgical operation on the target area within the inflated abdominal cavity.

Locking of the platform in the levitation magnetic field can be achieved in different processes. For example, a manual locking process may be performed following the insertion of the platform into the abdominal cavity while being held by the laparoscopic instrument the abdominal cavity is insufflated. After the platform is inserted and the cavity is inflated, the magnetic field is powered on and the operator moves the platform using the lap instrument in the abdominal space. The sensor captures the platforms location and displays to the operator where the locking point is. The display shows the necessary movement in order to locate the platform in locking position (up/down, forward/backwords). Once the platform is in place the display signals “locked” and the operator can gently let go of the platform which can stay levitated in the abdominal cavity.

An automatic locking process can also be used following the insertion of the platform into the abdominal cavity. After the insertion, the operator leaves the platform resting on the abdominal organs. The abdomen is then insufflated and the sensor is turned on without turning on the levitation magnetic field. The sensor senses the location of the platform and then two signals are sent to the computer controlling the movements: a first signal indicating the location of the platform and a second signal indicating the calculated locking point according to the lifter magnet complex location. The software then moves the lifter magnet complex and/or the OR table until the platform and the calculated locking point overlap. Following the operators approval the magnetic field is turned on and the platform is locked. The operator then elevates the platform (either by elevating the lifter magnet or lowering the OR table) and the platform stays levitated in the abdominal cavity.

In implementations of the described magnetic levitation systems in medical uses, different procedures for diagnosis and surgery may be used. In one example, instead of inserting a long tube (endoscope) through the mouth into the stomach which has some risks to the patient and discomfort, the patient can swallow this platform. A thread can be attached to the platform for later retrieval of the platform. Once the platform is inside the stomach, the levitation magnetic field is turned on to levitate the platform. Next, the video camera on the platform can be used to observe the inside of the stomach and biopsies or operations can be performed by the platform if needed. Upon completion of the procedure, the platform can be retrieved using the attached thread (rather than the large endoscope) that is inserted to the stomach via the mouth and grab the platform and pull it back to the mouth.

In another example, the magnetic levitation system is used to implement Natural Orifice Translumenal Endoscopic Surgery (NOTES), one type of Minimal Invasive Surgery operations. In this approach the surgery is done by inserting the magnetic platform through the mouth into the stomach and using the inserted platform to make an incision on the stomach wall from the inside. After the incision, the platform is pushed into the abdominal cavity like in laparoscopy. The platform can be miniaturized to allow for such incision and insertion. After the platform is pushed into the stomach and out to the abdominal cavity, the platform is then levitated and is controlled to move around inside the abdominal cavity to capture images and to perform surgical or diagnostic procedures.

One example of using a magnetic levitated system to perform a medical procedure can be conducted as the following: placing a patient on the surgical table to position the abdominal cavity in the surgical space; inflating the abdominal cavity with a gas; inserting the magnetic platform into the stomach through the patient's mouth; controlling a video camera in the magnetic platform to capture video images inside the stomach; and wirelessly transmitting the captured video images from the camera outside the patient's body to display on a display screen. Next, in this example, a surgical instrument mounted on the magnetic platform is wirelessly controlled to make an incision on a wall of the stomach. The magnetic platform is then moved out of the stomach through the incision into the inflated abdominal cavity and the magnet system is controlled to levitate the magnetic platform inside the inflated abdominal cavity. Subsequently, the video camera in the magnetic platform is operated to capture video images inside the inflated abdominal cavity and the captured video images from the camera are wirelessly transmitted outside the patient's body to display on a display screen. The relative position or orientation of the magnet system with respect to the surgical table is controlled to move the levitated magnetic platform inside the inflated abdominal cavity to search for a target area based on the displayed video images on the display screen. A surgical instrument mounted on the levitated magnetic platform is wirelessly controlled to perform a surgical operation on the target area within the inflated abdominal cavity. In the above, after completion of the surgical operation, the magnetic platform may be moved from the inflated abdominal cavity into the stomach through the incision and may be subsequently retrieved from the stomach through the patient's mouth.

While this specification contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few implementations are disclosed. However, it is understood that variations and enhancements may be made.

1. A magnetic levitation medical system, comprising:
a frame structured to define a surgical space that accommodates a surgical table for holding a patient, the magnetic frame comprising a top frame part above the surgical space;
a magnet system comprising (1) a plurality of lifting magnets mounted to the top frame part to produce a static magnetic field within the surgical space below the top frame part to exert a magnetic lifting force on a magnetic material against the gravity, and (2) at least one electromagnet mounted to the top frame part to produce an adjustable magnetic field in the surgical space;
a plurality of magnetic field sensors mounted to the frame to measure the magnetic field in the surgical space;
a magnetic platform formed of a magnetic material and configured to be magnetically levitated by the magnet system to levitate in the surgical space without a mechanical attachment and without a communication cable, the magnetic platform comprising at least one of a diagnostic probe that performs a measurement and a surgical tool that performs a surgical operation; and
a feedback control module to receive sensor signals from the magnetic field sensors and, in response to the sensor signals, to control the magnet system to adjust the magnetic field in the surgical space to stabilize and to control a position and motion of the levitated magnetic platform.

2. The system as in claim 1, wherein at least one of the magnetic field sensors is mounted to the top frame part.

3. The system as in claim 1, wherein:
the lifting magnets comprise permanent magnets.

4. The system as in claim 3, wherein:
the permanent magnets are permanent rare earth magnets.

5. The system as in claim 1, wherein:
the lifting magnets are electromagnets.

6. The system as in claim 1, wherein:
the magnet system and the magnet platform are configured to levitate the magnetic platform away from a surface of the magnet system by more than 5 inches.

7. The system as in claim 1, comprising:
a positioning module to control at least one of a relative position and a relative orientation of the frame with respect to the surgical table to control and adjust at least one of a position and an orientation of the suspected magnetic platform relative to the surgical table.

8. The system as in claim 1, wherein:
the magnet platform comprises an imaging device which captures images of a target near which the magnet platform is levitated and wirelessly transmits captured images and,
wherein the system further comprises a TV monitor to receive the captured images and to display the captured images.

9. The system as in claim 8, wherein:
the imaging device is a video camera to capture a live video of the target.

10. The system as in claim 8, wherein:
the imaging device comprises two spatially separated imaging sensors.

11. The system as in claim 10, comprising:
an imaging processor that receives the captured images from the two imaging sensors and processes the captured images from the two imaging sensors to produce a 3-dimensional image on the TV monitor.

12. The system as in claim 1, wherein:
the magnetic platform further comprises a light source to illuminate a target.

13. The system as in claim 1, comprising:
an operator control module operable to wirelessly communicate with the magnetic platform and to wirelessly control the at least one of the diagnostic probe and the surgical tool.

14. The system as in claim 13, wherein:
the operator control module comprises a joystick which operates to allow a user to manually adjust the joystick to control an operation of the magnetic platform.

15. The system as in claim 1, wherein:
the magnet platform comprises a DC power supply to supply electric power to an instrument on the magnetic platform.

16. The system as in claim 1, wherein:
the feedback control module comprises:
a plurality of electronic signal paths to respectively receive the sensor signals, each electronic signal path comprising at least one signal amplifier and at least one signal filter and operable to produce a respective processed sensor signal; and
a plurality of sampling circuits respectively connected to the electronic signal paths, each sampling circuits operable to average and hold each respective processed sensor signal, and
wherein the feedback control module applies output signals from the sampling circuits to control the magnet system to stabilize the levitated magnetic platform.

17. The system as in claim 1, wherein:
the feedback control module comprises:
an analog-to-digital conversion unit to convert the sensor signals into digital sensor signals that indicate at least a position of the levitated magnetic platform;
a digital signal processor to digitally process the digital sensor signals to produce digital control signals; and
a digital-to-analog conversion module to cover the digital control signals into analog control signals which are applied to control the magnet system to stabilize the levitated magnetic platform.

18. The system as in claim 1, wherein:
the feedback control module controls an amplitude of an electric current that drives the at least one electromagnet.

19. The system as in claim 1, wherein:
the feedback control module controls an amplitude of an electric current that drives the at least one electromagnet at a constant and varies a pulse width of the electric current.

20. The system as in claim 1, wherein:
the magnetic field sensors are Hall probes.

21. The system as in claim 1, wherein:
the diagnostic probe or the surgical tool is a MEMS device.

22. The system as in claim 1, wherein:
the frame further comprises a bottom frame part that is spaced from the top frame part to enclose the surgical space between the top frame part and the bottom frame part.

23. The system as in claim 22, wherein:
the magnet system comprises:
a plurality of additional lifting magnets mounted to the bottom frame part to produce an additional static magnetic field within the surgical space below the top frame part to exert an additional magnetic lifting force on the magnetic material against the gravity, and
at least one additional electromagnet mounted to the bottom frame part to produce an additional adjustable magnetic field in the surgical space.

24. The system as in claim 23, wherein:
at least one of the magnetic field sensors is mounted to the bottom frame part.

25. The system as in claim 23, wherein:
the frame comprises at least one side frame part that is positioned vertically below the top frame part and at one side of the surgical space, and
the magnet system comprises at least one electromagnet mounted to the side frame part to exert a side adjustable magnetic field in the surgical space to provide an additional control over the levitation of the magnetic platform.

26. The system as in claim 1, wherein:
the frame comprises at least one side frame part that is positioned vertically below the top frame part and at one side of the surgical space, and
the magnet system comprises at least one electromagnet mounted to the side frame part to exert a side adjustable magnetic field in the surgical space to provide an additional control over the levitation of the magnetic platform.

27. A method for using the system in claim 1 to perform a medical procedure in a patient's stomach, comprising:
attaching the magnetic platform to a cable;
having the patient swallow the magnetic platform into the stomach while leaving one end of the cable outside the patient's mouth with the other end of the cable attached to the swallowed magnetic platform;
positioning the patient on the surgical table so that the swallowed magnetic platform inside the surgical region of the magnet system;
controlling the magnet system to levitate the swallowed magnetic platform inside the stomach;
controlling a video camera in the magnetic platform to capture video images inside the stomach;
wirelessly transmitting the captured video images from the camera outside the patient's body to display on a display screen;
moving a relative position or orientation of the magnet system with respect to the surgical table to move the levitated magnetic platform inside the stomach to search for a target area based on the displayed video images on the display screen; and
pulling the cable attached to the swallowed magnetic platform to retrieve the magnetic platform from the stomach.

28. The method as in claim 27, comprising:
prior to pulling the cable to retrieve the magnetic platform, wirelessly controlling a biopsy instrument mounted on the levitated magnetic platform to remove a piece of tissue or sample liquid from the target area.

29. The method as in claim 27, comprising:
prior to pulling the cable to retrieve the magnetic platform, wirelessly controlling a surgical instrument mounted on the levitated magnetic platform to perform a surgical operation on the target area.

30. A method for using the system in claim 1 to perform a medical procedure, comprising:
placing a patient on the surgical table to position the abdominal cavity in the surgical space;
inflating the abdominal cavity with a gas;
inserting the magnetic platform into the stomach through the patient's mouth;
controlling a video camera in the magnetic platform to capture video images inside the stomach;
wirelessly transmitting the captured video images from the camera outside the patient's body to display on a display screen;
wirelessly controlling a surgical instrument mounted on the magnetic platform to make an incision on a wall of the stomach;
moving the magnetic platform out of the stomach through the incision into the inflated abdominal cavity;
controlling the magnet system to levitate the magnetic platform inside the inflated abdominal cavity;
operating the video camera in the magnetic platform to capture video images inside the inflated abdominal cavity;
wirelessly transmitting the captured video images from the camera outside the patient's body to display on a display screen;
moving a relative position or orientation of the magnet system with respect to the surgical table to move the levitated magnetic platform inside the inflated abdominal cavity to search for a target area based on the displayed video images on the display screen; and
wirelessly controlling a surgical instrument mounted on the levitated magnetic platform to perform a surgical operation on the target area within the inflated abdominal cavity.

31. The method as in claim 30, further comprising:
after completion of the surgical operation, moving the magnetic platform from the inflated abdominal cavity into the stomach through the incision; and
subsequently retrieving the magnetic platform from the stomach through the patient's mouth.

32. A method for operating a magnetically levitated platform to conduct a surgical operation within an abdominal cavity of a patient, comprising:
providing a magnet system to produce a variable magnetic field that defines a magnetic levitation region above a surgical table to levitate a magnetic platform and to control a position and motion of the magnetic platform;
placing the patient on the surgical table to position the abdominal cavity in the magnetic levitation region;
inflating the abdominal cavity with a gas;
inserting the magnetic platform inside the inflated abdominal cavity to levitate the magnetic platform;
controlling the variable magnetic field in the magnetic levitation region to levitate the magnetic platform in the inflated abdominal cavity and to stabilize the magnetic platform;
using a camera on the magnetic platform to capture one or more images of inside the inflated abdominal cavity including the target;
wirelessly transmitting the one or more captured images from the camera outside the patient's body to display on a display screen;
moving a relative position or orientation of the magnet system with respect to the surgical table to move the levitated magnetic platform near a target area within the inflated abdominal cavity; and
wirelessly controlling a surgical instrument mounted on the levitated magnetic platform to perform a surgical operation on the target area within the inflated abdominal cavity.

33. The method as in claim 32, comprising:
mounting the magnet system on an adjustable frame; and
moving the adjustable frame to move the levitated magnetic platform within the inflated abdominal cavity.

34. The method as in claim 32, comprising:
fixing the magnet system in position; and
moving the surgical table relative to the magnet system to move the levitated magnetic platform within the inflated abdominal cavity.

35. The method as in claim 32, comprising:
mounting the magnet system on an adjustable frame; and
moving both the adjustable frame and the surgical table to move the levitated magnetic platform within the abdominal cavity.

36. A magnetic levitation system, comprising:
a frame comprising a plurality of magnets to produce a variable magnetic field;
a plurality of magnetic field sensors mounted to the frame to measure the magnetic field;
a magnetic platform formed of a magnetic material and configured to be magnetically levitated by the magnetic field, the magnetic platform comprising a video camera to capture video images and a wireless communication unit to wirelessly transmit the video images outside the magnetic platform; and
a feedback control module to receive sensor signals from the magnetic field sensors and, in response to the sensor signals, to control the magnets to adjust the magnetic field to levitate and to stabilize the magnetic platform.

37. A method of operating the system in claim 36, comprising:
levitating the magnetic platform inside a confined space; and
using the video images from the camera to monitor the confined space.

38. The method as in claim 37, further comprising:
controlling a probe instrument mounted on the levitated magnetic platform to measure a parameter inside the confined space.

39. The method as in claim 38, wherein:
the measurement of the parameter is to a detect a hazard condition inside the confined space.

40. The method as in claim 38, wherein:
the confined space is within a person's body; and
the measurement of the parameter is to perform a medical diagnosis.

41. The method as in claim 37, wherein:
the confined space is within a person's body; and
the method further comprising:
controlling a surgical instrument mounted on the levitated magnetic platform to perform a surgical procedure.
